(19) United States
(12) Patent Application Publication
(10) Pub. No.: US 2007/0105186 A1
(43) Pub. Date: May 10, 2007

(54) Method for preserving microbial cells

(76) Inventors: Berman Cecil Gibson, Lexington, KY (US); Gerald Lynn Chrisope, Lake Charles, LA (US)

Correspondence Address: WARNER J DELAUNE JR;ADAMS AND REESE LLP, 450 LAUREL STREET, SUITE 1900, BATON ROUGE, LA, 70801, US

(21) Appl. No.: 11/542,063

(22) Filed: Oct. 3, 2006

Related U.S. Application Data
(63) Continuation-in-part of application No. 11/347,334, filed on Feb. 3, 2006, now abandoned.
(60) Provisional application No. 60/593,737, filed on Feb. 9, 2005.

(51) Int. Cl. C12Q 1/04; C12N 1/20; C12M 3/00

(52) U.S. Cl. 435/34; 435/252.3; 435/309.1

(57) ABSTRACT

A method of preserving microbial cells is provided, comprising dispensing microbial cells into preservation medium to produce a microbial cell suspension, impregnating a swab with a predetermined amount of the microbial cell suspension, and desiccating the impregnated swab. In one embodiment, the swab comprises a first end and a second end, wherein the second end comprises a network of fibers. In another embodiment, the method further comprises the steps of attaching the first end of the desiccated swab to a cap, inserting the desiccated swab into a tubular container, and sealing the tubular container with the cap.

### CROSS REFERENCES TO RELATED APPLICATIONS

This application is a continuation in part of application Ser. No. 11/347,334, filed Feb. 3, 2006 which is entitled to the benefit of provisional App. Serial No. 60/593,737, filed Feb. 9, 2005.

### BACKGROUND OF THE INVENTION

I. Field of the Invention

The present invention generally relates to a method of desiccating microbial cells. In particular, the present invention is useful for preserving, transferring, and recovering viable microbial cells. However, loss of viability is a constant concern and problems arise in the storage of viable microbial cells, especially when stored for extended periods of time.

II. Prior Art

A variety of preserved microorganisms are available commercially. Commercial provision of microorganisms on a global basis requires that the preserved microbial cells maintain viability throughout the rigors imposed by storage, distribution and shipping. In addition, microbial cells must remain viable for prolonged periods of subsequent storage at the final destination. Desiccation by freeze-drying or lyophilization is widely known and recognized as an effective method of preserving microbial cells. Detailed descriptions of lyophilization or freeze-drying methods for a variety of microorganisms are described in American Type Culture Collection Methods, I. Laboratory Manual on Preservation: Freezing and Freeze-Drying, Hatt, H. (ed.), ATCC (1980).

Lyophilization or freeze-drying involves the removal of water by sublimation from a frozen culture. If sufficient bound or unbound water is not removed during the preservation process, stability is severely compromised resulting in the loss of viable microbial cells over time. Insufficient removal of bound and unbound water results in residual water that enables metabolic processes to continue in the preserved cells. This results in the accumulation of metabolites, cell death and ultimately, a decreased shelf life.

There are additional problems associated with the various methods currently available for desiccating microorganisms. The available products generally comprise resealable storage bottles or vials containing discs or pellets of freeze-dried microorganisms. See for example U.S. Pat. Nos. 6,057,151 and 5,155,039. Such devices present a safety hazard due to the risk of injury from broken glass. In addition, they require a rehydration step before the microorganisms can be transferred to the appropriate culture media. Another available product, disclosed in U.S. Pat. No. 5,279,964 utilizes a plastic loop for storing and transferring preserved microorganisms. That product also requires a rehydration step where the loop must be dipped into a liquid before it is applied to the appropriate growth medium. Moreover, the currently available products do not address the problem of loss of viable microbial cells over time. For the foregoing reasons, more desirable methods of preservation are needed.

### SUMMARY OF THE INVENTION

It is an object of the present invention to reduce the loss of viable microbial cells that have been preserved, thereby improving stability and prolonging shelf life.

It is a further object of the present invention to eliminate the necessity of the rehydration step when recovering microbial cells that have been preserved by desiccation.

It is a further object of the present invention to improve recovery of desiccated microbial cells.

Therefore, a method in accordance with the present invention comprises dispensing microbial cells into a preservation medium to produce a microbial cell suspension, impregnating a swab with a predetermined amount of the microbial cell suspension, and desiccating the impregnated swab. In one embodiment the swab has one end that includes a network of synthetic fibers. The network of fibers is impregnated with the microbial cell suspension and then desiccated. The desiccated swab and a desiccant are inserted into a tubular container, which is substantially free from water and oxygen, and the container is sealed with a cap. In another embodiment, the desiccated swab, enclosed in the tubular container, is inserted and sealed in a foil pouch that is substantially free from water and oxygen.

In order to accomplish the goal of more effectively desiccating the microbial cells, the swab is preferably impregnated with microbial cells, which have been suspended in a preservation medium that includes, charcoal, skim milk, deionized water, trehalose and polyhydric alcohol. In a more specific embodiment the preservation medium may also include an additive selected from the group consisting of oxygen removing enzymatic compounds, horse serum, ascorbic acid and mixtures thereof. Optionally, the preservation medium may also include cryoprotectants such as glucose, sucrose, lactose, monosodium glutamate, bovine serum albumin, or glycol.

### BRIEF DESCRIPTION OF THE DRAWINGS

FIG. 1 depicts a side view of the preferred embodiment of the invention showing a swab sealed in a tubular container with a desiccant.

FIG. 2 depicts a detailed view of the fibrous network impregnated with the microbial cell suspension.

FIG. 3 depicts a view of the invention enclosed in a pouch with a desiccant.

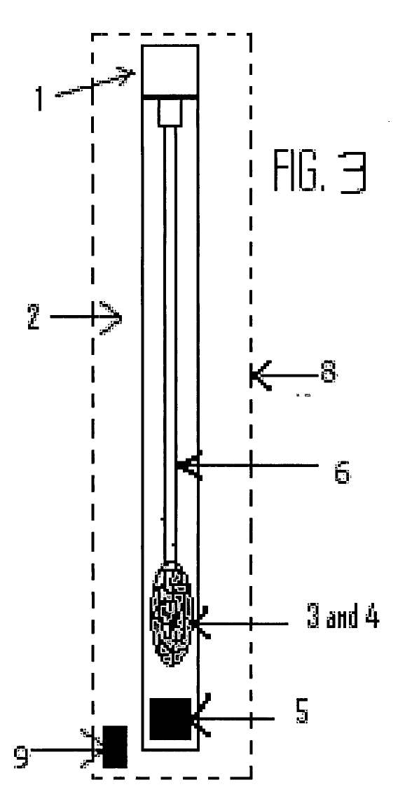

### DETAILED DESCRIPTION OF THE PREFERRED EMBODIMENTS

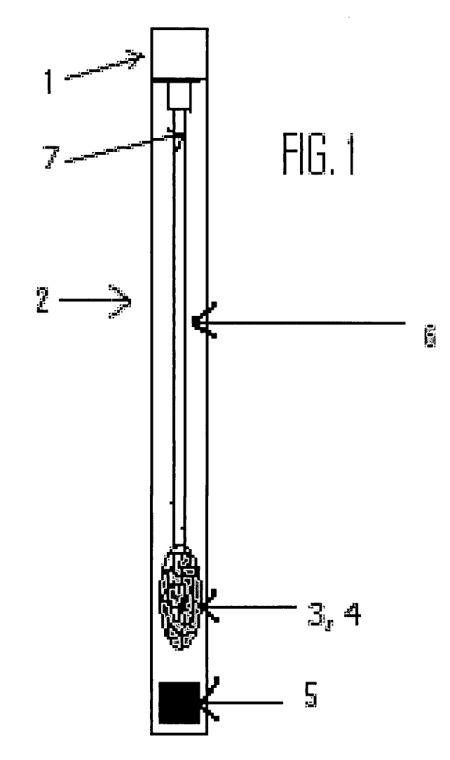

FIG. 1 illustrates a preferred embodiment of a fully assembled preservation system generally comprising a swab 6 having a fibrous tip 3 having a network of fibers and a non-fibrous end 7. The fibrous tip 3 is impregnated with a desiccated microbial cell suspension 4. The non-fibrous end 7 is operatively attached to a cap 1, which cap 1 is sealable attached to the open end 8 of a tube 2 or similar closable container, a desiccant 5 resides within tube 2 to preserve the environment for the cell suspension 4. The desiccant 5 is preferable a molecular sieve desiccant known to those in the art. Prior to assembly of the preservation system, the water and oxygen are removed from the tube 2 in accordance with the methods described herein.

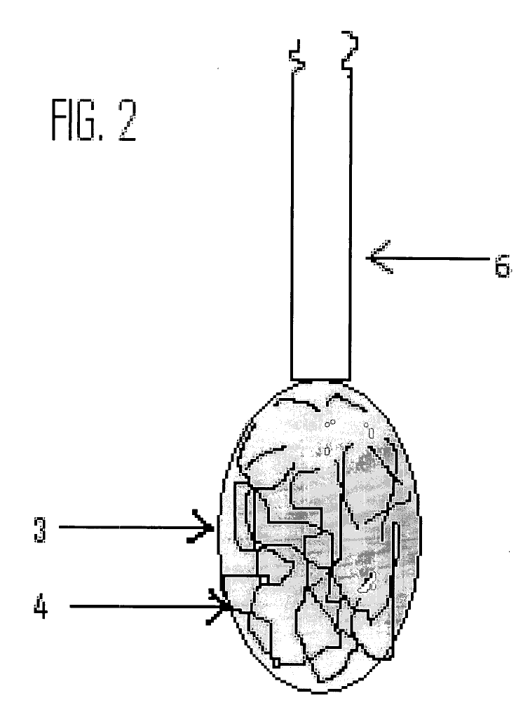

FIG. 2 depicts a detailed view of the fibrous network tip 3 of the swab, which is impregnated with microbial cell suspension 4. It is preferable for the fibers in the fibrous network 3 to be Dacron(g, but polyester, nylon, rayon, or other synthetic fibers may also be used with equivalent results. In the preferred embodiment, microbial cell suspension 4 is preserved in a preservation medium preferably comprising a mixture of polyhydric alcohol, charcoal, skim milk, deionized water, and trehalose. Polyhydric alcohols such as inositol and xylitol aid in supporting the microbial cell wall as water is removed. Optionally, the preservation medium may also include cryoprotectants such as glucose, sucrose, lactose, monosodium glutamate, bovine serum albumin, or glycol. The desired microbial cells are added to the preservation medium and vigorously agitated to produce the microbial cell suspension 4. When preserving anaerobic microbial cells, the preservation medium also includes preferably includes an oxygen removing enzymatic compound such as Oxyrase®, a product manufactured by Oxyrase, Inc. In a more preferred embodiment, horse serum and ascorbic acid are added to the preservation medium just prior to the introduction of the microbial cells. The preservation medium provides protection for the microbial cells during the preservation process and further aids in maintaining the viability of cells during subsequent storage.

After the microbial cells are introduced into the preservation medium to produce the microbial cell suspension 4, a pre-determined amount of the microbial cell suspension 4 is aliquoted with a pipet into a sterile microtiter plate. The fibrous network 3 of the swab 6 is impregnated by absorbing the aliquoted cell suspension 4. In the preferred embodiment, the fibrous network 3 is impregnated with 1-500 μL of microbial cell suspension 4. The impregnated swabs 6 undergo lyophilization in a VirTis Freeze Dryer or similar device, using the recipe shown in Table V. After the lyophilization process, the swabs 6 are removed from the freeze dryer and the non-fibrous end 7 is attached to the cap 1 by an adhesive. Before the swab 6 is inserted into the tubular container 2, a desiccant 5 is inserted and the container 2 is purged of most of the water and oxygen by purging it with zero grade nitrogen from a tank/nozzle system. After, the swab 6 is inserted into the tubular container 2, it is sealed with the cap 1. In the preferred embodiment the sealed tubular container 2 is finally inserted into a foil water-barrier pouch 8 containing a desiccant 9. The pouch 8 is purged with nitrogen gas, and is substantially free from water and oxygen when it is sealed. The preserved microbial cells are recovered or reconstituted by placing the swab 6 in direct contact with a solid or liquid culture media, and there is no need for a rehydration step.

It is the belief of the applicant that incorporation of the microbial cell suspension 4 throughout a fibrous network 3 provides a physical environment that allows greater removal of water during lyophilization thereby providing a method with improved stability and recovery of viable microbial cells. It is further the belief of the applicant that the lyophilization process creates a negative pressure which in turn creates a conduit of channels that surround each fiber and that the hydrophobicity of polyester fibers repels water augmenting creation of conduit-like channel network. That network of channels serves as a pipeline for the removal of bound and free water from the microbial cell suspension. The same channels that facilitate the removal of water, also facilitate recovery of the preserved microbial cells by allowing water in during the recovery step.

As can be seen for the foregoing description of the preferred and alternate embodiments, the present invention is intended to provide a method for preserving microbial cells that improves stability, lengthens shelf-life, improves recovery of the cells, and alleviates the rehydration step required by other methods.

### EXPERIMENTAL EXAMPLES

The following examples are provided in order to demonstrate and further illustrate certain preferred embodiments and aspects of the present invention and are not to be construed as limiting the scope thereof.

### Example 1

The preservation media was prepared with 5.0 g inositol, 1.0 g charcoal, 10 g skim milk, and 100 ml deionized water. The pH was adjusted to 7.0±0.2. The media was autoclaved at 105 C for 15 minutes, and cooled. A solution containing 10.0 g trehalose and 20 ml of deionized water was sterilized by filter sterilization with a 0.2 μ filter, mixed with the autoclaved solution and then dispensed into vials containing 10 or 20 mls. Colonies of microbial cells were collected from growth plates with a sterile loop and deposited into the vials of preservation media. The vials were vortexed for a minimum of five seconds. Using a pipet, 100 μL were aliquoted into each well of a sterile 96 well microtiter plate. The sterile swabs were allowed to soak up the aliquoted suspension and were covered with cellulose sterilization wrap.

The recipe in Table V was followed for lyophilization of the impregnated swabs using a VerTis Freeze Dryer. After removing the swabs from the freeze dryer, they were attached to the caps with a hot glue gun. The swabs were inserted into tubular containers that were purged with nitrogen gas. Molecular sieve desiccants were placed in the containers before they were sealed. Each container was placed into a foil pouch that had been purged with nitrogen gas. The foil pouches were heat sealed.

The preserved microbial cells were stored at 35-37 degrees C. for 28 days. No rehydration fluid was used for recovery. Preserved microbial cells were recovered from the swabs by direct inoculation of the fibrous network to culture media plates. The microbial cells were able to withstand the constant stress temperature for 28 days with only a 1-2 log reduction of colony forming units. The results are depicted in Table I.

### Example 2

In this example, microbial cells were preserved according to the methods described in Example 1. The preserved microbial cells were stored at 30 degrees C. for up to 6 months. Preserved microbial cells were recovered from the swabs by direct inoculation of the fibrous network to culture media plates. The microbial cells were able to withstand the constant room temperature conditions for up to 6 months days and maintain easy recovery without pre-rehydration. The results are depicted in Table II.

### Example 3

In this example, microbial cells were preserved according to the methods described in Example 1. The preserved microbial cells were stored at 2-8 degrees C. for up to 15 months. Preserved microbial cells were recovered from the swabs by direct inoculation of the fibrous network to culture media plates. The microbial cells were able to withstand the constant refrigerated temperature conditions for up to 15 months days and maintain easy recovery without pre-rehydration. The results are depicted in Table III.

### Example 4

A comparison study was conducted to evaluate the method of the present invention versus typical pellet structures for preservation of microbial cells. Microbial cells were recovered from the swabs by direct inoculation of the fibrous network to culture media plates; no rehydration fluid was necessary for recovery of viable cells. Microbial cells from the pellets were recovered by rehydration with 0.4 mL of the appropriate growth medium and subsequent four quadrant streak onto culture media plates. The methods of the current invention provided greater recovery of viable cells when compared to desiccated pellets. The results are depicted in Table IV.

TABLE I
ACCELERATED STUDIES (Storage Temperature: 35-37 C.)

| Organism | Initial (CFU'S/mL) | 28 days (CFU's/mL) |
|---|---|---|
| Aspergillus niger | 10⁴ | 10⁴ |
| Bacillus cereus | 10⁶ | 10⁵ |
| Burkholderia cepacia | 10⁸ | 10⁶ |
| Candida albicans | 10⁶ | 10⁵ |
| Haemophilus influenzae | 10⁶ | 10⁵ |
| Pseudomonas aeruginosa | 10⁷ | 10⁵ |
| Staphylococcus aureus | 10⁸ | 10⁷ |
| Staphylococcus epidermidis | 10⁷ | 10⁶ |
| Streptococcus bovis | 10⁷ | 10⁵ |
| Streptococcus pyogenes | 10⁷ | 10⁵ |
| Streptococcus pneumoniae | 10⁶ | 10⁴ |

Interpretation:
Test results reported are based on “dry” streak methods (no rehydration fluid utilized).
Conclusions:
Device allows product to withstand constant stress temperature for up to 28 days and maintain viability with only a 1-2 log reduction.

TABLE II
ACCELERATED STUDIES (STORAGE
TEMPERATURE: 30 C.)

| | | 1 | 2 | 4 | 6 |
|---|---|---|---|---|---|
| Organism | Initial | Month | Months | Months | Months |
| Streptococcus pyogenes | 4+ | 4+ | 4+ | 4+ | 3+ |
| Streptococcus aglactiae | 4+ | 4+ | 4+ | 4+ | 3+ |
| Escherichia coli | 4+ | 4+ | 4+ | 4+ | 3+ |
| Bacillus subtilis | 4+ | 4+ | 4+ | 4+ | 3+ |
| Staphylococcus aureus | 4+ | 4+ | 4+ | 4+ | 4+ |
| Haemophilus | 4+ | 4+ | 4+ | 4+ | 2+ |
| influenzae | | | | | |
| Streptococcus | 4+ | 4+ | 4+ | 4+ | 2+ |
| pneumoniae | | | | | |
| Enterococcus faecalis | 4+ | 4+ | 4+ | 4+ | 3+ |
| Klebsiella pneumonie | 4+ | 4+ | 4+ | 4+ | 3+ |
| Rhodococcus equi | 4+ | 4+ | 4+ | 4+ | 2+ |

Interpretation:
Viability Scale: 0 (No Growth), 1+ Growth in 1^{st} Quadrant, 2+ Growth in 2^{nd} Quadrant, 3+ Growth in 3^{rd} Quadrant, 4+ Growth in 4^{th} Quadrant.
Conclusions:
Device allows product to withstand constant room temperature conditions for up to 6 months and maintain easy recovery without pre-rehydration.

TABLE III
(STORAGE TEMPERATURE: 2-8 C.)

| | | 1 | 5 | 9 | 15 |
|---|---|---|---|---|---|
| Organism | Initial | Month | Months | Months | Months |
| Streptococcus pyogenes | 4+ | 4+ | 4+ | 4+ | 3+ |
| Streptococcus aglactiae | 4+ | 4+ | 4+ | 3+ | 3+ |
| Escherichia coli | 4+ | 4+ | 4+ | 3+ | 3+ |
| Bacillus subtilis | 4+ | 4+ | 4+ | 4+ | 3+ |
| Staphylococcus aureus | 4+ | 4+ | 4+ | 3+ | 4+ |
| Haemophilus | 4+ | 4+ | 4+ | 3+ | 3+ |
| influenzae | | | | | |
| Streptococcus | 4+ | 4+ | 3+ | 3+ | 3+ |
| pneumoniae | | | | | |
| Enterococcus faecalis | 4+ | 4+ | 4+ | 3+ | 3+ |
| Klebsiella pneumonie | 4+ | 4+ | 4+ | 3+ | 3+ |
| Rhodococcus equi | 4+ | 4+ | 3+ | 2+ | 2+ |

Interpretation:
Viability Scale: 0 (No Growth), 1+ Growth in 1^{st} Quadrant, 2+ Growth in 2^{nd} Quadrant, 3+ Growth in 3^{rd} Quadrant, 4+ Growth in 4^{th} Quadrant.
Conclusions:
Device allows product to withstand constant refrigerated temperature conditions for up to 15 months and maintain easy recovery without pre-rehydration.

TABLE IV
COMPARISON STUDY STORAGE TEMPERATURE (2-8 C.)

| Organism | Format | 12 Months |
|---|---|---|
| Escherichia coli | Pellet | 2+ |
| 25922 | Fibrous Network | 3+ |
| Streptococcus pneumoniae | Pellet | 2+ |
| 49150 | Fibrous Network | 3+ |
| Staphylococcus aureus | Pellet | 2+ |
| 25923 | Fibrous Network | 3+ |
| Bacillus cereus | Pellet | 2+ |
| 11778 | Fibrous Network | 3+ |
| Campylobacter jejuni | Pellet | 0 |
| 33291 | Fibrous Network | 2+ |

Interpretation:
Viability Scale: 0 (No Growth), 1+ Growth in 1^{st} Quadrant, 2+ Growth in 2^{nd} Quadrant, 3+ Growth in 3^{rd} Quadrant, 4+ Growth in 4^{th} Quadrant.
Conclusions:
Fibrous network provides for improved recovery of viable cells versus a lyophilized pellet. Particularly with Campylobacter jejuni.

TABLE V
Recipe # 0008

| | | |
|---|---|---|
| Temp | Time | Ramp/Hold |

Thermal Treatment Steps

| | | |
|---|---|---|
| Step # 1 | 0 | 0 |
| Step # 2 | 0 | 0 |
| Step # 3 | 0 | 0 |
| Step # 4 | 0 | 0 |
| Step # 5 | 0 | 0 |
| Step # 6 | 0 | 0 |
| Step # 7 | 0 | 0 |
| Step # 8 | 0 | 0 |
| Step # 9 | 0 | 0 |
| Step # 10 | 0 | 0 |
| Step # 11 | 0 | 0 |
| Step # 12 | 0 | 0 |

| | |
|---|---|
| Freeze Temp | −40° C. |
| Additional Freeze | 120 min |
| Condenser Setpoint | −40° C. |
| Vacuum Setpoint | 200 mTorr |

Primary Drying Steps

| | | | |
|---|---|---|---|
| Step # 1 | −45 | 480 | H |
| Step # 2 | −35 | 120 | H |
| Step # 3 | −25 | 120 | H |
| Step # 4 | −15 | 120 | H |
| Step # 5 | 0 | 120 | H |
| Step # 6 | 25 | 180 | H |
| Step # 7 | 0 | | |
| Step # 8 | 0 | | |
| Step # 9 | 0 | | |
| Step # 10 | 0 | | |
| Step # 11 | 0 | | |
| Step # 12 | 0 | | |
| Step # 13 | 0 | | |
| Step # 14 | 0 | | |
| Step # 15 | 0 | | |
| Step # 16 | 0 | | |
| Post Heat | 25 | | H |

| | |
|---|---|
| Secondary Temperature | O° C. |

What is claimed is:

1. A method for preserving microbial cells, comprising:
a. dispensing microbial cells into a preservation medium to produce a microbial cell suspension;
b. impregnating a swab with a predetermined amount of the microbial cell suspension; and
c. desiccating the impregnated swab.

2. The method of claim 1, wherein the preservation medium comprises polyhydric alcohol, charcoal, skim milk, deionized water, and trehalose.

3. The method of claim 2, wherein the polyhydric alcohol comprises inositol.

4. The method of claim 2, wherein the preservation medium further comprises an additive selected from the group consisting of oxygen removing enzymatic compound, horse serum, ascorbic acid and mixtures thereof.

5. The method of claim 1, wherein the preservation medium comprises one or more cryoprotectants selected from the group consisting of: glucose, sucrose, lactose, monosodium glutamate, bovine serum albumin, and glycol.

6. The method of claim 1, wherein the swab comprises a first end and a second end, wherein the second end comprises a network of synthetic fibers.

7. The method of claim 5, wherein the network of fibers comprises polyester.

8. The method of claim 1, wherein the pre-determined amount of the microbial cell suspension is between 1 μL and 500 μL.

9. The method of claim 1, wherein the pre-determined amount of the microbial cell suspension is between 75 μL and 125 μL.

10. The method of claim 1, wherein the step of desiccating comprises lyophilization.

11. The method of claim 1, wherein the step of desiccating comprises freeze-drying.

12. The method of claim 1, further comprising the steps of attaching the first end of the desiccated swab to a cap, inserting the desiccated swab into a tubular container, and sealing the tubular container with the cap.

13. The method of claim 11, wherein the tubular container is substantially free from water and oxygen.

14. The method of claim 11, wherein the tubular container contains a desiccant.

15. The method of claim 13, wherein the desiccant comprises molecular sieve desiccant.

16. The method of claim 1 1, further comprising the steps of inserting the sealed tubular container into a foil pouch and sealing the pouch.

17. The method of claim 15, wherein the foil pouch is substantially free from water and oxygen.

18. A method of culturing the microbial cells preserved in accordance with the method of claim 11 comprising removing the cap and the swab from the tubular container and placing the swab in direct contact with a culture medium.

19. A product for preserving microbial cells, comprising:
a. a swab having a first end and a second end, wherein said first end includes a fibrous tip adapted to receive a microbial cell suspension;
b. a cap operatively attached to said second end of said swab;
c. a tubular container adapted to receive said swab and said cap, wherein said tubular container includes a desiccant, and wherein said cap sealably encloses said swab within said tubular container.

20. The product of claim 18, wherein the fibrous tip comprises polyester.

21. The product of claim 18, wherein the microbial cell suspension comprises a preservation medium and microbial cells.

21. The product of claim 20, wherein the preservation medium comprises polyhydric alcohol, charcoal, skim milk, deionized water, and trehalose.

22. The method of claim 21, wherein the polyhydric alcohol comprises inositol.

23. The product of claim 21, wherein the preservation medium further comprises an additive selected from the group consisting of oxygen removing enzymatic compound, horse serum, ascorbic acid and mixtures thereof.

24. The product of claim 21, wherein the preservation medium comprises one or more cryoprotectants selected from the group consisting of: glucose, sucrose, lactose, monosodium glutamate, bovine serum albumin, and glycol.

25. The product of claim 18, wherein the desiccant comprises molecular sieve desiccant.

26. The product of claim 18, wherein the tubular container is substantially free from water and oxygen.
